(19) United States
(12) Patent Application Publication
(10) Pub. No.: US 2010/0104769 A1
(43) Pub. Date: Apr. 29, 2010

(54) AUTOMOTIVE COATING SURFACE ENHANCEMENT USING A PLASMA TREATMENT TECHNIQUE

(76) Inventors: John E. Boisseau, Bloomfield Hills, MI (US); Donald H. Campbell, Hartland, MI (US); Paul Deskovitz, Woodhaven, MI (US); Patrick J. Mormile, Birmingham, MI (US)

Correspondence Address: HOWARD & HOWARD ATTORNEYS PLLC;BASF CORPORATION, 450 West Fourth Street, Royal Oak, MI, 48067, US

(21) Appl. No.: 12/257,126

(22) Filed: Oct. 23, 2008

(51) Int. Cl. H05H 1/24; A61N 5/00

(52) U.S. Cl. 427/569; 250/492.3

(57) ABSTRACT

A technique of treating an automotive coating to modify surface-specific physical properties thereof while retaining bulk physical properties of the automotive coating includes the step of generating a plasma discharge in a plasma generating assembly. The automotive coating is treated with the plasma discharge. The automotive coating is completely cured prior to treatment with the plasma discharge. To treat the automotive coating, either 1) a precursor material is introduced into the plasma discharge to form a thin film on the automotive coating, or 2) the plasma discharge is free of a precursor material and weak bonds are destroyed on or near the surface of the automotive coating.

### BACKGROUND OF THE INVENTION

1. Field of the Invention

The present invention relates to treatment of polymer films using a plasma treatment technique and, more specifically, relates to treatment of automotive coating surfaces using atmospheric pressure generated plasma discharges.

2. Description of the Related Art

A plasma discharge is an electrically conductive gas containing charged particles. When atoms of the gas are excited to high energy levels, the atoms become ionized, thereby producing the plasma discharge containing electrically charged particles, i.e., ions and electrons. The plasma discharge is generated by introducing an inert gas, such as argon, nitrogen, or helium, between two electrodes and passing an electrical current between the two electrodes to excite the gas to the high energy levels.

A precursor material may be introduced into the plasma discharge. Depending upon the particular technique, the plasma discharge may be utilized to provide a source of free radicals to the precursor material, thereby triggering free radical polymerization of molecules in the precursor material, or the plasma discharge may be utilized to provide a source of heat, which may melt the precursor material into a molten form for deposition on the substrate.

Plasma discharges have been employed in the field of coatings to perform various functions. For example, plasma discharges have been used to deposit precursor materials on substrates through chemical vapor deposition (CVD) and sputtering techniques to thereby form thin films on the substrates. For CVD techniques, the precursor material is in vapor form and is introduced into the inert gas prior to excitation or into the plasma discharge. After exposure to the plasma discharge, the precursor material is deposited onto a substrate to form a thin film thereon. For sputtering techniques, one of the electrodes is a consumable anode, which is used as the source of the precursor material.

The plasma discharges have also been used to cure coating compositions that cure through dual-cure mechanisms, with one of the curing mechanisms being free-radical polymerization. Plasma discharges have also been used to activate surfaces of polymeric substrates for the purpose of enhancing wetting and improving adhesion between the substrate and the subsequently applied coatings, especially when the substrate surface is non-functionalized or non-polar. When the plasma discharges are used to cure the coating compositions or to activate the surfaces of polymeric substrates, no precursor material is typically used and the plasma discharge is employed to expose the coating composition or the surface of the polymeric substrate to a source of electrically charged particles, which electrically charged particles initiate free-radical polymerization or modify polymeric molecules on the surface of the polymeric substrate.

Automotive coatings, in particular, have many properties that must be fulfilled for successful overall performance. The various properties that must be fulfilled include, but are not limited to, scratch resistance, acid etch resistance, UV light resistance (weathering), chip resistance, solvent resistance, and various adhesion properties for purposes of achieving MVSS adhesion to stationary glass. Some of the properties are surface-specific and are required only within the top few microns or less of the automotive coating. Other properties must be satisfied throughout a bulk of the automotive coating. In many cases, compromises must be made in selection of specific automotive coatings between achievement of the necessary surface-specific physical properties and the physical properties required throughout the bulk of the automotive coatings.

Plasmas discharges have been used, in limited capacity, to halogenate polymeric molecules at the surface of automotive coatings. More specifically, a halogen is used as a precursor material, and exposure of the automotive coating surface to the plasma discharge including the halogen results in halogenation of polymeric molecules on the automotive coating surface, thereby producing a halogen containing polymer on the automotive coating surface and providing the automotive coating surface with properties of the halogen containing polymer.

In view of the foregoing, there remains a further opportunity to utilize plasma discharges to treat automotive coatings in a manner that enables achievement of desired surface-specific physical properties while retaining bulk physical properties of the automotive coatings.

### SUMMARY OF THE INVENTION

The present invention provides a technique of treating an automotive coating to modify surface-specific physical properties thereof while retaining bulk physical properties of the automotive coating. The technique comprises the step of generating a plasma discharge in a plasma generating assembly. The automotive coating is treated with the plasma discharge. The automotive coating is completely cured prior to treatment with the plasma discharge. To treat the automotive coating, either 1) a precursor material is introduced into the plasma discharge to form a thin film on the automotive coating, or 2) the plasma discharge is free of a precursor material and weak bonds are destroyed on or near the surface of the automotive coating.

By either forming the thin film on the automotive coating or destroying weak bonds on or near the surface of the automotive coating, desired surface-specific physical properties of the automotive coating are modified while bulk physical properties of the automotive coating is retained.

### DETAILED DESCRIPTION OF THE INVENTION

The instant invention provides various techniques of treating automotive coatings with a plasma discharge to modify surface-specific physical properties thereof while retaining bulk physical properties of the automotive coatings. The techniques can be categorized as either 1) utilization of a plasma discharge to deposit precursor materials onto automotive coatings, thereby forming thin films on the automotive coatings to modify surface-specific physical properties of the automotive coatings, or 2) exposing the automotive coatings to a plasma discharge to modify the surface of the automotive coatings themselves, thereby modifying surface-specific physical properties of the automotive coatings.

The automotive coating that is treated with the plasma discharge may be disposed on a basecoat layer on a substrate, optionally with additional layers such as a primer layer, electrocoat layer, etc. disposed beneath the basecoat layer. Alternatively, the automotive coating that is treated with the plasma discharge may be disposed directly on the substrate in the absence of any other layers and, in such embodiments, may include pigment. As such, the automotive coating that is treated with the plasma may be referred to as a clearcoat layer or a topcoat layer, depending upon whether or not pigment is present in the automotive coating. The substrate may be formed from any type of material, but is typically formed from metal or plastic. A common substrate may be a metal panel or a plastic part of a vehicle. Suitable metals that may be used to form the substrate include iron, steel, and alloys thereof; and aluminum, zinc, titanium, magnesium and alloys thereof; and any combination of the metals set forth herein.

Suitable plastics that may be used to form the substrate can include any thermoplastic or thermoset synthetic material known in the art, including fiber reinforced thermoset and thermoplastic materials. Non-limiting examples of thermoplastic materials that are suitable for use to form the substrates include polyethylene, polypropylene, thermoplastic polyolefins (“TPO”) such as polyethylene, polypropylene, reaction injected molded polyurethane (“RIM”), thermoplastic polyurethane (“TPU”), polyamides such as nylon, thermoplastic polyesters, acrylic polymers, vinyl polymers, polycarbonates, acrylonitrile-butadiene-styrene (“ABS”) copolymers, ethylene propylene diene terpolymer (“EPDM”) rubber, and combinations thereof. Non-limiting examples of thermoset materials that are suitable for use to form the substrates include polyesters, epoxides, phenolics, acrylics, and thermosetting polyurethanes such as “RIM” thermoset materials, and mixtures of any of the foregoing. Non-limiting examples of suitable thermoplastic materials include mixtures of any of the foregoing.

The automotive coating comprises the reaction product of at least a resin component and a crosslinking agent that is reactive with the resin component. Because treatment of the automotive coating in accordance with the instant invention is intended to modify surface-specific physical properties of the automotive coating, the automotive coating is completely cured prior to treatment with the plasma discharge. Stated differently, the treatment of the automotive coatings with the plasma discharge in accordance with the instant invention is not intended to cure the automotive coating to be treated, but is rather employed to modify automotive coatings that are completely formed and cured.

The resin component used in the present invention may be selected from the group of polymers, oligomers, materials, and combinations thereof. Suitable polymers and oligomers for purposes of the present invention include, but are not limited to, those having at least three monomeric units and/or those having a number average molecular weight of at least 1500 Daltons. In the context of the instant application, an oligomer is a compound containing in general on average from 2 to 15 monomer units. A polymer, in contrast, is a compound containing on average at least 10 monomer units. A material, for purposes of the present invention, is a compound or mixture of compounds that is not derived from monomeric units.

Non-limiting examples of polymers that are suitable for purposes of the present invention include acrylic resins, carbamate resins, polyester resins, polyurethane resins, vinyl resins, polycarbonate resins, epoxy resins, polysiloxane resins, and combinations thereof. Non-limiting examples of oligomers that are suitable for purposes of the present invention include the simple reaction products of a di-isocyanate with a functionalization agent such as a hydroxy acid, hydroxy carbamate, and hydroxy acrylate. Non-limiting examples of materials that are suitable for purposes of the present invention include fatty acids, dimers and trimers of fatty acids, didecanoic acid, and combinations thereof. In some cases, the same reactants used to form a polymer can also be used to from the oligomer or material, such as with some alkyd based resins. The resin may also include mixtures of polymers, oligomers, and materials, as alluded to above. Specific examples of resins that are suitable for purposes of the present invention are dual-hydroxy carbamate-functional acrylate resins that are disclosed in U.S. Pat. Nos. 6,858,693, 6,855,789, 6,696,535, 6,696,159, and/or 6,531,560. Additional resins that are suitable for purposes of the present invention are described in additional detail below in the context of a dual cure coating composition that may be used in accordance with one embodiment of the instant invention.

As also alluded to above, the resin component is reactive with the crosslinking agent, and thus includes one or more functional groups that are reactive with the crosslinking agent. The specific functional groups of the resin component may include, for example, active hydrogen donors such as hydroxyl functional groups, amino functional groups, acid functional groups, carbamate and urea functional groups, amide functional groups, activated methylene functional groups, and combinations thereof. The functional groups of the resin component may alternatively include active hydrogen acceptor groups such as anhydride functional groups, epoxy functional groups, activated aminoplast functional groups, free or blocked isocyanate functional groups, cyclic carbonate functional groups, silane functional groups, and combinations thereof. The functional groups of the resin component may alternatively include groups that can undergo addition reactions such as activated vinyl groups including acrylate functional groups and the combination pair of isocyanurate with epoxy. The specific functional groups of the resin component depend on the specific functional groups of the crosslinking agent, as described below. Further, the resin component can have a mixture of the above types of functional groups provided that any reactivity between the different groups can be controlled, i.e., so long as the mixture of the above types of functional groups does not hurt the storage stability of the resin component. Typically, the resin component has functional groups that are active hydrogen acceptor groups and/or functional groups that can undergo addition reactions.

The functional group or groups of the resin component may be masked or blocked in such a way so that they become unblocked and available for reaction with the crosslinking agent under desired curing conditions, such as at elevated temperatures.

The resin component is typically present in a curable coating composition, prior to reaction with the crosslinking agent, in an amount of at least 1 part by weight solids, more typically in an amount of from about 1 to about 70 parts by weight solids based on 100 parts by weight of all solids in the curable coating composition.

Typically, when the resin component has active hydrogen donor groups, the crosslinking agent has active hydrogen acceptor groups such as those described above as suitable for the resin component. Particularly suitable crosslinking agents that include the active hydrogen acceptor groups, for purposes of the present invention, are aminoplasts. The aminoplasts comprise the reaction product of an aldehyde with an activated amine with or without additional etherification. Non-limiting examples of activated amines are amines connected to aromatic rings, such as benzene, melamine, benzoquatamine; primary carbamates; urea; amides; vinyl amines; and combinations thereof. However, it is to be appreciated that when the resin component includes active hydrogen acceptor groups, the crosslinking agent may include active hydrogen donor groups. As set forth above in the context of the resin component, in some cases, mixtures of functional groups can be used. For example, in one embodiment, the resin component may include acid functional groups, hydroxy functional groups, carbamate functional groups, and/or acrylic functional groups. In this example, the crosslinking agent may include aminoplast functional groups, isocyanate functional groups, silane functional groups, epoxy functional groups, and/or acrylic functional groups. The reaction between the functional groups of the resin component and the functional groups of the crosslinking agent can be activated by heat and/or UV light. Suitable cross-linking agents for purposes of the present invention are selected from the group of blocked polyisocyanates, blocked polyisocyanurates, polycarboxylic acid halides, aminoplast resins, and combinations thereof. As appreciated by those skilled in the art, an aminoplast resin is formed by the reaction product of formaldehyde and an amine where the preferred amine is a urea or a melamine. In other words, the aminoplast resin may include urea resins and melamine-formaldehyde resins. Additional crosslinking agents that are suitable for purposes of the present invention are described in additional detail below in the context of the dual cure coating composition.

The crosslinking agent is typically present in the curable coating composition, prior to reaction with the resin component, in an amount of at least 5 parts by weight solids, more typically in an amount of from about 10 to about 50 parts by weight solids, most typically in an amount of from about 15 to about 25 parts by weight solids, based on 100 parts by weight of all solids in the curable coating composition.

The curable coating composition may further include a catalyst for catalyzing the reaction between the resin component and the crosslinking agent. Suitable catalysts for purposes of the present invention may be selected from the group of tin catalysts, acid catalysts, acid phosphates, aromatic acids, and combinations thereof. Specific examples of suitable tin catalysts include dibutyltin diacetate (DBTDA) and dibutyltin dilaurate (DBTDL). Specific examples of suitable acid catalysts include sulfonic acids including dodecylbenzene sulfonic acid (DDBSA), dinonylnapthalene sulfonic acid (DNNSA), dinonylnapthalene disulfonic acid (DNNDSA); and p-toluene sulfonamine (PTSA). Additional suitable catalysts are described below in the context of the dual cure coating composition. The catalyst is typically present in the curable coating composition, prior to the reaction between the resin component and the crosslinking agent, in an amount of from 0.1 to 3 parts by weight solids, more typically from 1 to 2 parts by weight solids, based on 100 parts by weight of all solids in the curable coating composition.

The curable coating composition may be utilized, for example, in the form of a substantially solid powder, as a liquid that does not require additional solvent, or as a dispersion. When the curable coating composition is in the form of a dispersion, a solvent is typically used. Suitable solvents act as a solvent with respect to both the resin component and the crosslinking agent. In general, as known in the art, the solvent may be any of a number of organic solvent(s), including water, depending on the solubility characteristics of the resin component and the crosslinking component in the curable coating composition. In one embodiment, the solvent is a polar organic solvent. The polar solvent may be a polar aliphatic solvent or polar aromatic solvent, such as a ketone, ester, acetate, aprotic amide, aprotic sulfoxide, or aprotic amine. Examples of useful solvents include methyl ethyl ketone, methyl isobutyl ketone, m-amyl acetate, ethylene glycol butyl ether-acetate, propylene glycol monomethyl ether acetate, xylene, n-methylpyrrolidone, or blends of aromatic hydrocarbons. In another embodiment, the solvent is water or a mixture of water with small amounts of aqueous co-solvents. Suitable co-solvents include acetates such as butyl acetate, hexyl acetate, and octyl acetate; glycol ethers and glycol ether acetates, such as propylene glycol ether and propylene glycol monomethyl ether acetate; and ketones, such as methyl propyl ketone, methyl isobutyl ketone, and methyl hexyl ketone. Glycol ethers and glycol ether acetates are especially preferred. Further, the solvent may include non-polar aromatic and/or aliphatic solvents. Additional solvents that are suitable for purposes of the present invention are described in additional detail below in the context of the dual cure coating composition.

The solvent may be present in the curable coating composition, prior to the reaction between the resin component and the crosslinking agent, in an amount of from about 10 to about 60 parts by weight, more typically in an amount of from about 30 to about 50 parts by weight, based on 100 parts by weight of the curable coating composition.

As mentioned above, in one embodiment, the coating composition is the “dual cure” coating composition. As defined herein, “dual cure” refers to curable coating compositions that require exposure to both actinic radiation and thermal energy to achieve a degree of crosslinking and achieve desired performance properties. Thus, in one aspect, the dual cure coating compositions are at least partially curable or polymerizable upon exposure to some portions of the electromagnetic radiation spectrum. In another aspect of the disclosure, the dual cure coating compositions are at least partially thermally curable or polymerizable upon exposure to thermal or heat energy.

Radiation cure and thermal cure may occur sequentially or concurrently. In one embodiment, the dual cure coating compositions are subjected to a first stage of curing followed by a second stage of curing. Either radiation cure or thermal cure may occur first. Typically, the dual cure coating composition is first subjected to actinic radiation, especially UV radiation, followed by a second stage of cure, wherein the dual cure coating composition previously subjected to actinic radiation is subjected to a thermal cure.

It is to be appreciated that the second stage cure need not immediately succeed the first stage and can occur after the application of one or more subsequently applied coating compositions. For example, it is to be appreciated that one or more additional coating compositions may be applied to the radiation cured coating, and the one or more additionally applied coatings may then be simultaneously thermally cured together with the radiation cured coating composition.

Actinic radiation as used herein refers to energy having wavelengths of less than 500 nm and corpuscular radiation such as electron beam. Preferred actinic radiation will have wavelengths of from 180 to 450 nm, i e, in the UV region. More preferably, the actinic radiation will be UV radiation having wavelengths of from 225 to 450 nm. The most preferred actinic radiation will be UV radiation having wavelengths of from 250 to 425 nm.

Heat or thermal energy, as used herein, refers to the transmission of energy by either contact via molecular vibrations or by certain types of radiation.

Heat energy transferred by radiation as used herein refers to the use of electromagnetic energy generally described as infrared (IR) or near-infrared (NIR), i.e., energy having an approximate wavelength of from 800 nm to 10.sup.-3 m.

Heat as used herein also encompasses energy transferred via convection or conduction. Convection refers to the transmission of heat by the rise of heated liquids or gases and the fall of colder parts. Conduction may be defined as the transmission of matter or energy.

The dual cure coating compositions comprise at least four components: a radiation curable resin component (a1) that polymerizes upon exposure to actinic radiation, especially UV radiation, a thermally curable binder component (a2) that polymerizes upon exposure to heat, a thermally curable crosslinking component (a3) that has at least 2 isocyanate groups per molecule, and at least one additive (a4) for absorbing or otherwise preventing transmission of ultraviolet radiation.

The radiation curable resin component (a1) contains on average at least two functional groups per molecule, and more typically at least three functional groups. Typically, each functional group has at least one bond that is activatable upon exposure to actinic radiation, especially UV radiation, so as to crosslink. For example, in one embodiment, each functional group of the radiation curable resin component (a1) has one UV activatable bond.

Typically, the radiation curable resin component (a1) comprises less than or equal to six functional groups on average per molecule, and most typically less than or equal to five functional groups on average per molecule.

Examples of suitable bonds that can be activated with actinic radiation, and especially UV radiation, are carbon-hydrogen single bonds, carbon-carbon single bonds, carbon-oxygen single bonds, carbon-nitrogen single bonds, carbon-phosphorus single bonds, carbon-silicon single bonds, carbon-carbon double bonds, carbon-oxygen double bonds, carbon-nitrogen double bonds, carbon-phosphorus double bonds, carbon-silicon double bonds, or carbon-carbon triple bonds.

Highly suitable carbon-carbon double bonds are present, for example, in at least one of a (meth)acrylate group, an ethacrylate group, a crotonate group, a cinnamate group, a vinyl ether group, a vinyl ester group, an ethenylarylene group, a dicyclopentadienyl group, a norbornenyl group, a isoprenyl group, an isopropenyl group, an allyl group, a butenyl group, an ethenylarylene ether group, a dicyclopentadienyl ether group, a norbornenyl ether group, an isoprenyl ether group, an isopropenyl ether group, an allyl ether group, a butenyl ether group, an ethenylarylene ester group, a dicyclopentadienyl ester group, a norbornenyl ester group, an isoprenyl ester group, an isopropenyl ester group, an allyl ester group, and a butenyl ester group. It is to be appreciated that (meth)acrylics and (meth)acrylates refer to acrylates and methacrylates as well as acrylics and methacrylics.

The radiation curable resin component (a1) may further comprise at least one functional group that is reactive with the isocyanate groups of the thermally curable crosslinking component (a3).

Examples of suitable isocyanate-reactive groups include, but are not limited to, thiol groups, primary amino groups, secondary amino groups, imino groups, and hydroxyl groups.

The radiation curable resin component (a1) may further comprise at least one functional group that is a hydroxyl-reactive functional group. Examples of suitable hydroxyl-reactive groups include, but are not limited to, isocyanates, aminoplasts, epoxy groups, silane groups, cyclic anhydrides, and cyclic lactones.

The radiation curable resin component (a1) may be oligomeric or polymeric. In contrast, a low molecular mass compound in the context of the instant application refers to a compound that derives substantially from only one basic structure or monomer unit. Compounds of this kind may also be referred to as reactive diluents and are discussed below in regards to optional reactive diluent component (a5).

The radiation curable resin component (a1) generally has a number average molecular weight of from 500 to 50,000, more typically from 1000 to 5000. In one embodiment, the sum of radiation curable resin component (a1) and any optional reactive diluents (a5) may have a double bond equivalent weight of from 400 to 2000 g/mol, more typically from 500 to 900 g/mol. In addition, the combination of radiation curable resin components (a1) and any optional reactive diluents (a5) typically have a viscosity at 23° C. of from 250 to 11,000 mPas.

The radiation curable resin component (a1) may be employed in an amount of from 1 to 50% by weight, typically from 3 to 45% by weight, and more typically from 5 to 20% by weight, based in each case on the total nonvolatile solids of film-forming components of the dual cure coating composition. Film-forming components as used herein refers to components such as the radiation curable resin component (a1), thermally curable binder component (a2), thermally curable crosslinking component (a3), optional reactive diluent (a5), and any other monomeric, oligomeric or polymeric components that chemically react with any of components (a1), (a2), or (a3) so as to enter into the resulting polymerized network.

Specific examples of suitable radiation curable resin components (a1) include, but are not limited to, the oligomer and/or polymer classes of the (meth)acryloyl-functional (meth)acrylic copolymers, polyether acrylates, polyester acrylates, polyesters, epoxy acrylates, urethane acrylates, amino acrylates, melamine acrylates, silicone acrylates and phosphazene acrylates, the corresponding (meth)acrylates, vinyl ethers, and vinyl esters. Radiation curable resin component (a1) is typically free from aromatic structural units.

In one example, the radiation curable resin component (a1) comprises a urethane(meth)acrylate. Urethane(meth)acrylates suitable for use as the radiation curable resin component (a1) may be obtained by reacting a diisocyanate or a polyisocyanate with a chain extender that is at least one of a diol, a polyol, a diamine, a polyamine, a dithiol, a polythiol, and an alkanolamine, and then reacting the remaining free isocyanate groups with at least one hydroxyalkyl(meth)acrylate or a hydroxyalkyl ester of one or more ethylenically unsaturated carboxylic acids. The amounts of chain extenders, diisocyanates and/or polyisocyanates, and hydroxyalkyl esters in this case may be chosen so that 1) the ratio of equivalents of the isocyanate (NCO) groups to the reactive groups of the chain extender (hydroxyl, amino and/or mercaptyl groups) is between 3:1 and 1:2, and most typically 2:1, and 2) the hydroxyl (OH) groups of the hydroxyalkyl esters of the ethylenically unsaturated carboxylic acids are stoichiometric with regard to the remaining free isocyanate groups of the prepolymer formed from isocyanate and chain extender.

It is also possible to prepare urethane(meth)acrylates suitable for use as the radiation curable resin component (a1) by first reacting some of the isocyanate groups of a diisocyanate or polyisocyanate with at least one hydroxyalkyl ester and then reacting the remaining isocyanate groups with a chain extender. The amounts of chain extender, isocyanate, and hydroxyalkyl ester should also be selected such that the ratio of equivalents of the NCO groups to the reactive groups of the hydroxyalkyl ester is between 3:1 and 1:2, preferably 2:1, while the ratio of equivalents of the remaining NCO groups to the OH groups of the chain extender is 1:1.

Illustrative examples of urethane(meth)acrylates suitable for use as the radiation curable resin component (a1) include polyfunctional aliphatic urethane acrylates that are commercially available in materials such as CRODAMER® UVU 300 from Croda Resins Ltd., Kent, Great Britain; GENOMER® 4302, 4235, 4297, or 4316 from Rahn Chemie, Switzerland; EBECRYL® 284, 294, IRR 351, 5129, or 1290 from UCB, Drogenbos, Belgium; ROSKYDAL® LS 2989 or LS 2545 or V94-504 from Bayer AG, Germany; VIAKTIN® VTE 6160 from Vianova, Austria; or LAROMER® 8861 from BASF AG.

Hydroxyl-containing urethane(meth)acrylates suitable for use as the radiation curable component (a1) are disclosed in U.S. Pat. No. 4,634,602 A and U.S. Pat. No. 4,424,252 A. An example of a suitable polyphosphazene(meth)acrylate is the phosphazene dimethacrylate from Idemitsu, Japan.

As set forth above, the dual cure coating composition further comprises at least one thermally curable binder component (a2) comprising at least two isocyanate-reactive groups. Examples of suitable isocyanate-reactive groups are those described above with respect to the isocyanate-reactive groups of the radiation curable resin component (a1). Typically, the isocyanate reactive groups are hydroxyl groups.

At least 5% up to 100%, more typically from 20% to 40%, of the binder component (a2) by solids weight of the binder component (a2) is a component (X). Component (X) is a polymer with at least two isocyanate reactive functional groups, a glass transition temperature (Tg) of less than 0° C., and an equivalent weight of greater than 225 g/mol. Typically, the Tg of component (X) is less than −20° C., and more typically less than −50° C. Typically, the equivalent weight is greater than 265 g/mol. Typically, component (X) is at least one of a polyether diol, a polyether polyol, a polyester diol, and a polyester polyol.

Examples of suitable polyether diols for component (X) include, but are not limited to, polyoxyalkylenes such as polyethylene oxide, polypropylene oxide, and polytetrahydrofuran. Generally, there are at least 4 repeating or monomer units in the polyether diol, more typically from 7 to 50 repeating units.

Examples of suitable polyether polyols include, but are not limited to, the polyether polyols sold under the trademarks LUPRANOL®, PLURACOL®, PLURONIC®, and TETRONIC® from BASF; ARCOL®, DESMOPHEN®, and MULTRANOL® from Bayer; VORANOL® from Dow; CARPOL® from E. R. Carpenter; PORANOL® from Hannam, Korea; and KONIX® from Korea Polyol.

Examples of suitable polyester diols include, but are not limited to polylactones (such as poly(e-caprolactone)) and polyesters derived from dimer fatty acid, isophthalic acid, and 1,6-hexanediol. Suitable poly(e-caprolactone) is available as TONE® 201 or TONE® 301 from Dow Chemical. Generally, there are at least 4 repeating units in the polyester diol or triol, more typically from 4 to 50 repeating units. Examples of suitable polyester diols can be found in U.S. Pat. No. 5,610,224.

The polyester polyols may be formed through lactone extension of polyols having more than 3 hydroxyl groups. The polyester polyols can be prepared from low molecular weight alcohols and polybasic carboxylic acids such as adipic acid, sebacic acid, phthalic acid, isophthalic acid, tetrahydrophthalic acid, hexahydrophthalic acid, maleic acid, the anhydrides of these acids, and mixtures of these acids and/or acid anhydrides. Polyols suitable for the preparation of the polyester polyol include, but are not limited to, polyhydric alcohols such as ethylene glycol, propanediols, butanediols, hexanediols, neopentyl glycol, diethylene glycol, cyclohexanediol, cyclohexanedimethanol, trimethylpentanediol, ethylbutylpropanediol ditrimethylolpropane, trimethylolethane, trimethylolpropane, glycerol, pentaerythritol, dipentaerythritol, trishydroxyethyl isocyanate, polyethylene glycol, polypropylene glycol, and the like, as well as combinations of these. The polyol component may also include, if desired, minor amounts of monohydric alcohols, for example butanol, octanol, lauryl alcohol, and ethoxylated and propoxylated phenols. In another embodiment, a polyester polyol can be modified by reaction with a lactone. One specific example of a suitable polyester polyol is an e-caprolactone extension of pentaerythritol. Generally, there are at least on average 2 lactone monomer units, more typically from 2 to 25 lactone monomer units, per hydroxyl group on the polyol. Further examples of polyester diols can be found in U.S. Pat. Nos. 6,436,477 and 5,610,224.

While the at least one thermally curable binder component (a2) has at least two isocyanate-reactive groups, more than two isocyanate groups are also possible. In particular, the thermally curable binder component (a2) may have from two to ten isocyanate-reactive groups per molecule, most typically from two to seven isocyanate-reactive groups per molecule.

The thermally curable binder component (a2) is oligomeric or polymeric as defined above. Number average molecular weights of from 500 to 50,000 are suitable.

Oligomers and polymers generally suitable for use as the thermally curable binder component (a2) may be (meth)acrylate copolymers, polyesters, alkyds, amino resins, polyurethanes, polylactones, polyester polyols, polycarbonates, polyethers, epoxy resin-amine adducts, (meth)acrylate diols, partially saponified polyvinyl esters of polyureas, and mixtures thereof.

Polyesters having active hydrogen groups such as hydroxyl groups are especially suitable for use as thermally curable binder component (a2). Such polyesters may be prepared by the polyesterification of organic polycarboxylic acids (e.g., phthalic acid, hexahydrophthalic acid, adipic acid, maleic acid) or their anhydrides with organic polyols containing primary or secondary hydroxyl groups (e.g., ethylene glycol, butylene glycol, neopentyl glycol).

Suitable polyesters can be prepared by the esterification of a polycarboxylic acid or an anhydride thereof with a polyol and/or an epoxide. Suitable polycarboxylic acids used to prepare the polyester may comprise monomeric polycarboxylic acids or anhydrides thereof having 2 to 18 carbon atoms per molecule. Among the acids that are useful are phthalic acid, hexahydrophthalic acid, sebacic acid, and other dicarboxylic acids of various types. Minor amounts of monobasic acids can be included in the reaction mixture, for example, benzoic acid, stearic acid, acetic acid, and oleic acid. Also, higher carboxylic acids can be used, for example, trimellitic acid and tricarballylic acid. Anhydrides of the acids referred to above, where they exist, can be used in place of the acid. Also, lower alkyl esters of the acids can be used, for example, dimethyl glutarate and dimethyl terephthalate.

Polyols that can be used to prepare the polyester include diols such as alkylene glycols. Specific examples include ethylene glycol, 1,4-butanediol, 1,6-hexanediol, neopentyl glycol, and 2,2-dimethyl-3-hydroxypropyl-2,2-dimethyl-3-hydroxypropionate. Other suitable glycols include hydrogenated Bisphenol A, cyclohexanediol, cyclohexanedimethanol, caprolactone-based diols such as the reaction product of c-caprolactone and ethylene glycol, hydroxy-alkylated bisphenols, polyether glycols such as poly(oxytetramethylene)glycol, and the like. Although the polyol component can comprise all diols, polyols of higher functionality can also be used. Examples of polyols of higher functionality would trimethylolethane, trimethylolpropane, pentaerythritol, and the like.

Some thermally curable binders (a2) that may be suitable for use in the dual cure coating composition are commercially available under the trade names DESMOPHEN® 650, 2089, 1100, 670, 1200, or 2017 polyester polyols from Bayer, PRIPLAST® dimer based polyester polyols or PRIPOL® dimer fatty acid resins from Uniqema, Chempol®, polyester or polyacrylate-polyol from CCP, CRODAPOL® polyester polyol resins from Cray Valley, LTS polyester polyol adhesion resins from Creanova, or SETAL® 26-1615 from Nuplex of Louisville, Ky.

Typically, the thermally curable binder component (a2) is substantially free from functional groups having bonds activatable upon exposure to UV radiation. Such functional groups may be those as described above with regard to the functional group of the radiation curable resin component (a1). Typically, the thermally curable binder component (a2) is a fully saturated compound.

Optionally, the thermally curable component (a2) may be selected to have a polydispersity (PD) of less than 4.0, typically from 1.5 to less than 3.0. Polydispersity is determined from the following equation: (weight average molecular weight (M_{w})/number average molecular weight (Mₙ)). A monodisperse polymer has a PD of 1.0. Further, as used herein, Mₙ and M_{w} are determined from gel permeation chromatography using polystyrene standards.

In another optional aspect, the thermally curable binder component (a2) may also be selected so as to have less than 5% by weight of aromatic ring moieties, typically from 0 to 2% by weight of aromatic ring moieties, based on the nonvolatile weight of thermally curable binder component (a2).

The amount of component (a2) in the dual cure coating compositions may vary widely and is guided by the requirements of the individual case. However, thermally curable binder component (a2) is typically used in an amount of from 5% to 90% by weight, more typically from 9% to 50% by weight, based on the total nonvolatile solids of the film-forming components of the dual cure coating composition.

As set forth above, the dual cure coating composition further comprises at least one thermally curable crosslinking component (a3). Typically, the thermally curable crosslinking component (a3) is a blocked or unblocked di- and/or polyisocyanate.

The thermally curable crosslinking component (a3) may contain on average at least 2.0, typically more than 3.0 isocyanate groups on average per molecule and, while there is no limit to the number of isocyanate groups per molecule, typically less than or equal to 6.0 isocyanate groups per molecule. Typically, the thermally curable crosslinking component (a3) has from 2.5 to 3.5 isocyanate groups on average per molecule.

Examples of suitable diisocyanates are isophorone diisocyanate (i e., 5-isocyanato-i-isocyanatomethyl-1,3,3-trimethylcyclohexane), 5-isocyanato-1-(2-iso-cyanatoeth-1-yl)-1,3,3-trimethylcyclohexane, 5-iso-cyanato-1-(3-isocyanatoprop-1-yl)-1,3,3-trimethylcyclohexane, 5-isocyanato-(4-isocyanatobut-1-yl)-1,3,3-tri-methylcyclohexane, 1-isocyanato-2-(3-isocyanatoprop-1-yl)cyclohexane, 1-isocyanato-2-(3-isocyanatoeth-1-yl)cyclohexane, 1-isocyanato-2-(4-isocyanatobut-1-yl)cyclohexane, 1,2-diisocyanatocyclobutane, 1,3-di-isocyanatocyclobutane, 1,2-diisocyanatocyclopentane, 1,3-diisocyanatocyclopentane, 1,2-diisocyanatocyclohexane, 1,3-diisocyanatocyclohexane, 1,4-diisocyanatocyclohexane, dicyclohexylmethane-2,4-diisocyanate, trimethylene diisocyanate, tetramethylene diisocyanate, pentamethylene diisocyanate, hexamethylene diisocyanate (HDI), ethylethylene diisocyanate, trimethylhexane diisocyanate, heptamethylene diisocyanate, methylpentyl diisocyanate (MPDI), nonane triisocyanate (NTI) or diisocyanates derived from dimer fatty acids, as sold under the commercial designation DDI 1410 by Henkel and described in the patent publications WO 97/49745 and WO 97/49747, 2-heptyl-3,4-bis(9-isocyanatononyl)-1-pentylcyclbhexane, or 1,2-, 1,4-, or 1,3-bis(isocyanatomethyl)cyclohexane, 1,2-, 1,4-, or 1,3-bis(2-isocyanatoethyl)cyclohexane, 1,3-bis(3-isocyanatopropyl)cyclohexane, 1,2-, 1,4-, or 1,3-bis(4-isocyanatobutyl)cyclohexane or liquid bis(4-isocyanatocyclohexyl)methane with a trans/trans content of up to 30% by weight, as described in the patent applications DE 44 14 032 A1, GB 1220717 A1, DE 16 18 795 A1, and DE 17 93 785 A1, isophorone diisocyanate, 5-isocyanato-l-(2-isocyanatoethyl)-1,3,3-trimethylcyclohexane, 5-isocyanato-1-(3-isocyanatopropyl)-1,3,3-trimethylcyclohexane, 5-isocyanato-(4-isocyanatobutyl)-1,3,3-trimethylcyclohexane, 1-isocyanato-2-(3-isocyanatopropyi)cyclohexane, 1-isocyanato-2-(3-isocyanatoethyl)cyclohexane, 1-isocyanato-2-(4-isocyanatobut-1-yl)cyclohexane, or HDI.

Examples of suitable polyisocyanates are isocyanate-containing polyurethane prepolymers that can be prepared by reacting polyols with an excess of diisocyanates and that are typically of low viscosity.

It is also possible to use polyisocyanates containing isocyanurate, biuret, allophanate, iminooxadiazindione, urethane, urea, carbodiimide, and/or uretdione groups, prepared conventionally from the above-described diisocyanates. Examples of suitable preparation processes and polyisocyanates are known, for example, from the patents CA 2,163,591 A, U.S. Pat. No. 4,419,513, U.S. Pat. No. 4,454,317 A, EP 0 646 608 A, U.S. Pat. No. 4,801,675 A, EP 0 183 976 A1, DE 40 15 155 A1, EP0 303 150 A1, EP0 496 208 A1, EPO 524 500 A1, EPO 566 037 A1, U.S. Pat. No. 5,258,482 A1, U.S. Pat. No. 5,290,902 A1, EP 0 649 806 A1, DE 42 29 183 A1, and EP 0 531 820 A1, or are described in the published European patent application EP1122273 A3.

High-viscosity polyisocyanates described in German patent application DE 198 28 935 A1, or the polyisocyanate particles surface-deactivated by urea formation and/or blocking, as per the European patent applications EP 0 922 720 A1, EP 1 013 690 A1, and EP 1 029 879 A1 are also suitable for use as the thermally curable crosslinking component (a3).

Also suitable as the thermally curable crosslinking component (a3) are the adducts of polyisocyanates with dioxanes, dioxolanes and oxazolidines containing isocyanate-reactive functional groups and still containing free isocyanate groups, described in the German patent application DE 196 09 617 A1.

Aminoplast resins are also suitable for use as the thermally curable crosslinking component (a3). Examples of suitable aminoplast resins include melamine formaldehyde resin (including monomeric or polymeric melamine resin and partially or fully alkylated melamine resin including high imino melamines), urea resins (e.g., methylol ureas such as urea formaldehyde resin, alkoxy ureas such as butylated urea formaldehyde resin) and the like. Also useful are aminoplast resins where one or more of the amino nitrogens is substituted with a carbamate group for use in a process with a curing temperature below 150° C., as described in U.S. Pat. No. 5,300,328.

Examples of suitable tris(alkoxycarbonylamino)triazines are described in U.S. Pat. Nos. 4,939,213 and 5,084,541, and Eur. Pat. 0 624 577.

In one embodiment, the thermally curable crosslinking component (a3) is substantially free of functional groups having bonds activatable upon exposure to actinic radiation, especially UV radiation. Such bonds are described above in regards to functional groups of component (a1). In one specific example, the thermally curable crosslinking component (a3) is a polyisocyanurate of HDI that is substantially free of carbon-carbon double bonds.

The amount of thermally curable crosslinking component (a3) in the dual cure coating compositions is typically from 5% to 70% by weight, most typically from 25% to 45% by weight, based on the total nonvolatile content of the film-forming components of the dual cure coating composition.

In one embodiment, the ratio of isocyanate (NCO) groups of component (a3) to the sum of isocyanate-reactive functional groups in components (a1) and (a2) is less than 1.30, typically from 0.75 to 1.00.

As set forth above, the dual cure coating composition further comprises additives (a4) for absorbing or otherwise preventing transmission of ultraviolet radiation. Examples of suitable additives (a4) include ultraviolet light absorbers (UVA), light stabilizers, and blends of UVA and light stabilizers. Examples of suitable UVAs include benzophenones, benzotriazoles, triazines or benzoates, oxalanilides, and salicylates. Non-limiting examples of UVAs are TINUVIN 400®, TINUVIN 1130®, TINUVIN 328®, TINUVIN 234®, TINUVIN 1577®, and TINUVIN 384-2®, all produced by Ciba Specialty Chemicals.

Examples of suitable light stabilizers are hindered amine light stabilizers (HALS) and free-radical scavengers, generally derivatives of 2,2,6,6-tetramethyl piperidine. Non-limiting examples of HALS are TINUVIN 123®, TINUVIN 152®, and TINUVIN 292®, all produced by Ciba Specialty Chemicals.

As alluded to above, the dual cure coating compositions may further comprise a reactive diluent (a5) that is thermally curable and/or curable with actinic radiation. If used, reactive diluents (a5) are typically curable with actinic radiation. Typically, such reactive diluents further comprise one or more functional groups reactive with the thermally curable crosslinking component (a3). In one embodiment, the reactive diluent (a5) is curable with actinic radiation such as UV radiation and further comprises a plurality of functional groups reactive with isocyanate groups such as are described above with regards to functional groups of components (a1) and (a2).

Examples of suitable thermally curable reactive diluents (a5) are positionally isomeric diethyloctanediols or hydroxyl-containing hyperbranched compounds or dendrimers, as described in the patent applications DE 198 09 643 A1, DE 198 40 605 A1, and DE 198 05 421 A1.

Further examples of suitable reactive diluents (a5) are polycarbonatediols, polyesterpolyols, poly(meth)acrylate diols or hydroxyl-containing polyadducts.

Further examples of suitable reactive diluents (a5) include, but are not limited to, butyl glycol, 2-methoxypropanol, n-butanol, methoxybutanol, n-propanol, ethylene glycol monomethyl ether, ethylene glycol monobutyl ether, diethylene glycol monomethyl ether, diethylene glycol propanediol ether, diethylene glycol diethyl ether, diethylene glycol monobutyl ether, trimethylolpropane, ethyl 2-hydroxylpropionate or 3-methyl-3-methoxybutanol and also derivatives based on propylene glycol, e.g., ethoxyethyl propionate, isopropoxypropanol or methoxypropyl acetate.

Further examples of suitable reactive diluents (a5) that may be crosslinked with actinic radiation include (meth)acrylic acids and esters thereof, maleic acid and its esters, including monoesters, vinyl acetate, vinyl ethers, vinyl ureas, and the like. Examples that may be mentioned include alkylene glycol di(meth)acrylate, polyethylene glycol di(meth)acrylate, 1,3-butanediol di(meth)acrylate, vinyl(meth)acrylate, allyl (meth)acrylate, glycerol tri(meth)acrylate, trimethylolpropane tri(meth)acrylate, trimethylolpropane di(meth)acrylate, styrene, vinyl toluene, divinylbenzene, pentaerythritol, tri(meth)acrylate, pentaerythritol tetra(meth)acrylate, dipentaerythritol penta(meth)acrylate, propylene glycol di(meth)acrylate, hexanediol di(meth)acrylate, ethoxyethoxyethyl acrylate, N-vinylpyrrolidone, phenoxyethyl acrylate, dimethylaminoethyl acrylate, hydroxyethyl(meth)acrylate, butoxyethyl acrylate, isobornyl(meth)acrylate, dimethylacrylamide, dicyclopentyl acrylate, and the long-chain linear diacrylates described in EP 0 250 631 A1 with a molecular weight of from 400 to 4000, preferably from 600 to 2500. For example, the two acrylate groups may be separated by a polyoxybutylene structure. It is also possible to use 1,12-dodecylpropanediol and the reaction product of 2 moles of acrylic acid with one mole of a dimer fatty alcohol having generally 36 carbon atoms. Mixtures of the aforementioned monomers are also suitable.

Further examples of suitable reactive diluents (a5) curable with actinic radiation are those described in Rompp Lexikon Lacke und Druckfarben, Georg Thieme Verlag, Stuttgart, New York, 1998, on page 491 under the entry on “Reactive diluents”.

The dual cure coating compositions may also have one or more photoinitiators and typically have at least one photoinitiator. If the dual cure coating composition is to be crosslinked with UV radiation, a photoinitiator is typically used. When used, the photoinitiator is typically present in the dual cure coating composition in an amount of from 0.1% to 10% by weight, most typically from 0.5% to 5% by weight, based on the total solids content of the dual cure coating composition.

Examples of suitable photoinitiators are those of the Norrish II type, whose mechanism of action is based on an intramolecular variant of the hydrogen abstraction reactions as occur diversely in the case of photochemical reactions (by way of example, reference may be made here to Rompp Chemie Lexikon, 9.sup.th, expanded and revised edition, Georg Thieme Verlag, Stuttgart, Vol 4, 1991) or cationic photoinitiators (by way of example, reference may be made. here to Rompp Lexikon Lacke und Druckfarben, Georg Thieme Verlag, Stuttgart, 1998, pages 444 to 446), especially benzophenones, benzoins or benzoin ethers, or phosphine oxides. It is also possible to use, for example, the products available commercially under the names IRGACURE® 184, IRGACURE® 819, IRGACURE® 1800, and IRGACURE® 500 from Ciba Geigy, GENOCURE® MBF from Rahn, and LUCIRIN® TPO and LUCIRIN® TPO-L from BASF AG. Besides the photoinitiators, customary sensitizers such as anthracene may be used in effective amounts.

The dual cure coating compositions may also comprise at least one thermal crosslinking initiator that forms radicals at a temperature of from 80° C. to 120° C. Examples of thermal crosslinking initiators include thermolabile free-radical initiators such as organic peroxides, organic azo compounds or carbon-carbon cleaving initiators such as dialkyl peroxides, peroxocarboxylic acids, peroxodicarbonates, peroxide esters, hydroperoxides, ketone peroxides, azo dinitriles or benzpinacol silyl ethers. Such thermal initiators may be present in amounts of from 0 to 10% by weight, typically from 1 to 5% by weight, based on the total solids content of the dual cure coating composition.

The dual cure coating composition may further comprise water and/or at least one inert organic or inorganic solvent. Examples of inorganic solvents are liquid nitrogen and supercritical carbon dioxide. Examples of suitable organic solvents are the high-boiling (“long”) solvents or low boiling solvents commonly used in coatings, for example ketones such as methyl ethyl ketone, methyl isoamyl ketone, or methyl isobutyl ketone, esters such as ethyl acetate, butyl acetate, ethyl ethoxypropionate, methoxypropyl acetate, or butyl glycol acetate, ethers such as dibutyl ether, or ethylene glycol, diethylene glycol, propylene glycol, dipropylene glycol, butylene glycol, or dibutylene glycol dimethyl, diethyl, or dibutyl ether, N-methylpyrrolidone, or xylenes or mixtures of aromatic and/or aliphatic hydrocarbons such as SOLVENTNAPHTHA®, petroleum spirit 135/180, dipentenes or SOLVESSO® (cf. also “Paints, Coatings and Solvents”, Dieter Stoye and Werner Freitag (editors), Wiley-VCH, 2.sup.nd edition, 1998, pages 327 to 349).

The coating compositions used in accordance with the instant invention (including the dual cure coating composition described in detail above) may further comprise one or more pigments and/or fillers, especially when the coating compositions are used to form a topcoat. Suitable pigments and fillers for clearcoat and topcoat compositions are known in the art. The amount of the pigments and/or fillers in the coating compositions used in accordance with the instant invention may be from 0% to 50% by weight, most typically from 5% to 30% by weight, based on the total nonvolatile content of the coating composition.

The coating compositions may further optionally comprise one or more coating additives in effective amounts, i.e., in amounts of up to 40% by weight, and typically up to 10% by weight, based on the total solids content of the coating compositions. Examples of suitable coatings additives are crosslinking catalysts such as blocked sulfonic acid catalysts, dibutyltin dilaurate, or lithium decanoate; slip additives; polymerization inhibitors; defoamers; emulsifiers, especially nonionic emulsifiers such as alkoxylated alkanols and polyols, phenols, and alkylphenols, or anionic emulsifiers such as alkali metal salts or ammonium salts of alkane carboxylic acids, alkanesulfonic acids, and sulfo acids of alkoxylated alkanols and polyols, phenols, and alkylphenols; wetting agents such as siloxanes, fluorine compounds, carboxytic monoesters, phosphoric esters, polyacrylic acids, and their copolymers, polyurethanes or acrylate copolymers, which are available commercially under the tradename MODAFLOW® or DISPARLON®; adhesion promoters such as tricyclodecanedimethanol; leveling agents; film-forming auxiliaries such as cellulose derivatives; flame retardants; sag control agents such as ureas, modified ureas, and/or silicas, as described for example in the references DE 199 24 172 A1, DE 199 24 171 A1, EP 0 192 304 A1, DE 23 59 923 A1, DE 18 05 693 A1, WO 94/22968, DE 27 51 761 C1, WO 97/12945, and “Farbe+Lack”, November 1992, pages 829 ff.; rheology control additives, such as those known from the patents WO 94/22968, EP 0 276 501 A1, EP 0 249 201 A1, and WO 97/12945; crosslinked polymeric microparticles, as disclosed for example in EP 0 038 127 A1; inorganic phyllosilicates such as aluminum magnesium silicates, sodium magnesium phyllosilicates, and sodium magnesium fluorine lithium phyllosilicates of the montmorillonite type; silicas such as AEROSIL® silicas; or synthetic polymers containing ionic and/or associative groups such as polyvinyl alcohol, poly(meth)acrylamide, poly(meth)acrylic acid, polyvinylpyrrolidone, styrene-maleic anhydride or ethylene-maleic anhydride copolymers and their derivatives, or hydrophobically-modified.sub.1 ethoxylated polyurethanes or polyacrylates; flatting agents such as magnesium stearate; and/or precursors of organically modified, ceramic materials such as hydrolyzable organometallic compounds, especially of silicon and aluminum. Further examples of suitable coatings additives are described in the textbook “Lackadditive” [Additives for coatings] by Johan Bieleman, Wiley-VCH, Weinheim, New York, 1998.

One specific example of a curable coating composition that is suitable for forming the automotive coating treated in accordance with the instant invention is commercially available under the tradename DynaSeal® from BASF Corporation of Florham Park, N.J.

In one embodiment of the instant invention, the automotive coating may further comprise a morphing additive, i.e., an additive that is activated upon exposure to the plasma discharge, resulting in modification of the surface-specific properties of the automotive coatings. More specifically, when used, the morphing additives are included in the curable coating composition and, therefore, in the bulk of the automotive coating upon curing of the curable coating composition. However, activation of the morphing additives is required to bring about changes to the physical properties of the automotive coating that are attributable to the morphing additives. Activation of the morphing additives is accomplished through exposure of the automotive coating surface to the plasma discharge, resulting in modification of the surface-specific properties of the automotive coatings while the morphing additives remain inactivate throughout the bulk of the automotive coatings and thereby enable the automotive coatings to retain the desired bulk properties. When the morphing additives are included in the curable coating composition and activated to affect the surface-specific properties of the automotive coating, the surface-specific properties of the automotive coating are typically modified at a distance of from the surface of the automotive coating to a depth of less than five (5) microns. Suitable morphing additives may be selected from the group of silicones, fluoropolymers, polyesters, acrylics, and combinations thereof. Although specific amounts of the morphing additives may vary depending upon the type of morphing additive and the desired surface-specific physical property to be achieved, the morphing additives are typically present in the curable coating compositions in an amount of from 0.005% to 0.5%, more typically from 0.01% to 0.2%, most typically from 0.01% to 0.05% by weight based on the total weight of the curable coating compositions.

As set forth above, the automotive coating is treated with the plasma discharge in accordance with the instant invention. The plasma discharge is generated in a plasma generating assembly. While the instant invention is not limited to a particular manner of producing the plasma discharge, the plasma discharge is typically generated under conditions of atmospheric pressure. Atmospheric pressure plasma discharges are advantageous due to the fact that such systems do not require an enclosed space for facilitating plasma generation at vacuum pressure and, therefore, offer free ingress and egress of the workpieces/webs including the automotive coating into and out of the plasma discharge. Furthermore, commercially available plasma generating assemblies are versatile and may be mounted upon a robotic arm to enable selective control over regions of the automotive coating to be treated with the plasma discharge.

Atmospheric pressure plasma assemblies that are suitable for purposes of the instant invention are known in the art. In particular, the plasma generating assembly includes at least one pair of parallel electrodes spaced from each other, and the plasma discharge is generated by introducing an inert gas, such as argon, nitrogen, or helium, between the at least one pair of electrodes and passing an electrical current between the two electrodes to excite the gas to high energy levels that are necessary to form the plasma discharge. A precursor material may be introduced into the plasma discharge along with the inert gas, and the flow rate of the precursor material may vary depending upon the type of precursor material used. The precursor material may be mixed with the inert gas prior to introducing the inert gas between the two electrodes, and may be mixed prior to introducing the inert gas into the plasma generating assembly itself.

The plasma discharge generated in the plasma generating assembly is directed out of the plasma generating assembly through controlling the flow of the inert gas and, optionally, precursor material, between the electrodes. In this manner, the plasma discharge is typically directed out of the plasma generating assembly and onto the automotive coating to be treated. When the plasma discharge includes the precursor material, a thin film is formed on the automotive coating surface for purposes of modifying the surface-specific properties of the automotive coating. When the plasma discharge is free of precursor material, the automotive coating surface itself is modified by the plasma discharge.

As alluded to above, in one embodiment, the plasma discharge is used to deposit precursor materials onto the automotive coatings described above, thereby forming a thin film on the automotive coatings to modify surface-specific physical properties of the automotive coatings. The plasma discharge may be utilized to provide a source of free radicals to the precursor material, thereby initiating free radical polymerization of molecules in the precursor material, or the plasma discharge may be utilized to provide a source of heat, which may melt the precursor material into a molten form for deposition onto the automotive coating without exposing the automotive coating to excessively high temperatures that would damage the automotive coating. Examples of surface-specific properties that may be affected by forming the thin film on the automotive coating include scratch resistance, acid etch resistance, adhesion of glass to the automotive coating through an adhesive in accordance with motor vehicle safety standards (often referred to as MVSS adhesion), gloss of the automotive coating, and recoat adhesion for purposes of surface repair.

The automotive coating is treated with the plasma discharge including the precursor material for a sufficient period of time to form a thin film on the automotive coating having the desired film thickness (as described in further detail below).

The thin films formed on the automotive coatings typically have a thickness that is sufficient to affect the surface-specific property for which the thin film is intended to modify, but is also sufficiently thin to minimize detrimental effects to the properties of the automotive coating that are satisfied throughout a bulk of the automotive coating. The thickness of the thin film may be varied depending upon the particular precursor to be used and is generally from about 5 to about 600 nm. Further, the film thickness may vary depending upon the desired result to be achieved. For example, to impart low gloss features to the automotive coating, larger film thicknesses are typically employed within the ranges set forth above.

In another embodiment, as set forth above, the automotive coating is treated with the plasma discharge to modify the surface of the automotive coatings themselves, thereby modifying surface-specific physical properties of the automotive coatings. More specifically, in this embodiment, the plasma discharge is free from precursor materials and the plasma discharge provides a source of free radicals to the automotive coating itself. By providing the source of free radicals to the automotive coating, it is believed that the plasma discharge functions to destroy weak bonds, such as ether bonds, on or near the surface of the automotive coating that would otherwise be left in the automotive coating. The weak bonds are vulnerable to chemical attack and, when left in the automotive coatings, leave the automotive coatings vulnerable to acid etch over time. By destroying the weak bonds, stronger chemical bonds remain at or near the surface of the automotive coating, thereby providing an effective barrier against acid etching as well as providing resistance to scratching. As set forth in detail above, it is also possible to include the morphing additive in the curable coating composition that is cured to form the automotive coating, with the morphing additive providing the strong bonds at or near the surface of the automotive coating that provide the automotive coating with excellent acid etch resistance, scratch resistance, and/or other desirable physical properties.

As with the scenario described above when the morphing additives are included in the curable coating composition, when the automotive coating is treated with the plasma discharge, the surface-specific properties of the automotive coating are typically modified at a distance of from the surface of the automotive coating to a depth of about 5 microns, which is a sufficient depth to impart the surface-specific properties to the automotive coating while retaining the bulk properties of the automotive coating. Of course, it is to be appreciated that the depth at which the surface-specific properties are imparted to the automotive coating may vary depending upon many factors including length of time of plasma discharge exposure, inert gas flow rate, electrical current passed between the electrodes, distance of the plasma assemblies from the automotive coating, etc.

Automotive coatings treated in accordance with the instant invention have excellent etch resistance, as measure in accordance with ASTM D-7356 (400 hr), excellent scratch/mar resistance as measured in accordance with the Crockmeter method using 9 micron paper and, in some cases, exhibit noteworthy properties relative to gloss properties. For example, when plasma coating 2 is used to form the thin film on the automotive coating (see the Examples section below, including Table 1), etch ratings are often decreased by about one unit across various chemistries for the curable coating composition used to form the automotive coating as compared to similar untreated automotive coatings. Further, after scratch testing, automotive coatings including the thin film formed from plasma coating 2 retain in excess of 15% higher gloss than similar untreated automotive coatings and, in some instances, retain in excess of 20% higher gloss than similar untreated automotive coatings. Notably, gloss values measured for automotive coatings including the thin film formed from plasma coating 2 are artificially low. For example, for automotive coatings having the thin film and having a high-gloss jet-black appearance, low gloss values are obtained upon measurement with a gloss meter that correspond to a lower-gloss hazy or milky appearance. It is believed that the low gloss values are attributable to a light of light refraction phenomenon with the gloss meter as opposed to an actual visual effect of low gloss.

When the automotive coatings are treated with the plasma discharge in the absence of the precursor material, etch ratings are often decreased by up to two units across various chemistries for the curable coating composition used to form the automotive coating. Further, after scratch testing, automotive coatings treated with the plasma discharge in the absence of the precursor material often retain at least 10% higher gloss than similar untreated automotive coatings and, in some instances, retain at least 15% higher gloss than similar untreated automotive coatings.

The following examples are intended to illustrate, and not to limit, the instant invention.

### EXAMPLES

Various coating systems were prepared and treated with the plasma treatment techniques in accordance with the instant invention. In particular, substrates were coated with a basecoat composition to form a basecoat layer, which was allowed to cure. Upon cure of the basecoat layer, the coated substrates were then coated with different curable coating compositions to form clearcoat layers thereon. After completely curing the clearcoat layers, the clearcoat layers on the various substrates were subjected to various plasma treatment techniques. Table 1, below, sets forth the various Examples and Comparative Examples that were prepared and lists the type of clearcoat composition used, the plasma treatment technique used, and various processing parameters that were applied to the plasma treatment technique as well as resulting thin film thickness when applicable.

TABLE 1

| | | Plasma |
|---|---|---|
| Example | Clearcoat Composition | Treatment/Coating |
| Comp. Ex. 1 | Acrylic Melamine | No Plasma Treatment |
| Ex. 1 | Acrylic Melamine | Plasma Coating 1 |
| Ex. 2 | Acrylic Melamine | Plasma only |
| Ex. 3 | Si Modified Acrylic Melamine | Plasma Coating 2 |
| Ex. 4 | F Modified Acrylic Melamine | Plasma Coating 2 |
| Comp. Ex. 2 | Carbamate #1 | No Plasma Treatment |
| Ex. 5 | Carbamate #1 | Plasma Coating 1 |
| Ex. 6 | Carbamate #1 | Plasma only |
| Ex. 7 | Si Modified Carbamate #1 | Plasma Coating 2 |
| Ex. 8 | F Modified Carbamate #1 | Plasma Coating 2 |
| Comp. Ex. 3 | Carbamate #2 | No Plasma Treatment |
| Ex. 9 | Carbamate #2 | Plasma Coating 1 |
| Ex. 10 | Carbamate #2 | Plasma only |
| Ex. 11 | F Modified Carbamate #2 | Plasma Coating 2 |
| Comp. Ex. 4 | 2K Urethane | No Plasma Treatment |
| Ex. 12 | 2K Urethane | Plasma Coating 1 |
| Ex. 13 | 2K Urethane | Plasma only |
| Ex. 14 | Si Modified 2K Urethane | Plasma Coating 2 |
| Comp. Ex. 5 | Low Gloss Carbamate #3 | No Plasma Treatment |
| Ex. 15 | Low Gloss Carbamate #3 | Plasma Coating 1 |

Plasma coating 1 has a coating thickness that ranges between 50-90 nm with localized areas and individual flakes measuring up to 105 nm thick and comprises the polymerization product of a precursor material.

Plasma coating 2 has a coating thickness that ranges between 50-90 nm with localized areas and individual flakes measuring up to 105 nm thick and comprises the polymerization product of another precursor material.

Acrylic Melamine Clearcoat Composition is product code E126CM005T commercially available from BASF Corporation.

Si Modified Acrylic Melamine Clearcoat Composition is the Acrylic Melamine Clearcoat Composition described above including 0.05% by weight, based upon the total weight of the clearcoat composition, of Byk-306 polyether-modified polydimethylsiloxane commercially available from Byk-Chemie used as a morphing additive.

F Modified Acrylic Melamine Clearcoat Composition is the Acrylic Melamine Clearcoat Composition described above including 0.01% by weight, based upon the total weight of the clearcoat composition, of Fluorad FC-430 fluoroaliphatic polymeric ester commercially available from 3M Corporation and used as a morphing additive.

Carbamate #1 Clearcoat Composition is product code R10CG060S commercially available from BASF Corporation.

Si Modified Carbamate #1 Clearcoat Composition is the Carbamate #1 Clearcoat Composition described above including 0.05% by weight, based upon the total weight of the clearcoat composition, of Byk-306 used as a morphing additive.

F Modified Carbamate #1 Clearcoat Composition is the Carbamate #1 Clearcoat Composition described above including 0.01% by weight, based upon the total weight of the clearcoat composition, of Fluorad FC-430 used as a morphing additive.

Carbamate #2 Clearcoat Composition is product code R10CG062 commercially available from BASF Corporation.

F Modified Carbamate #2 Clearcoat Composition is the Carbamate #1 Clearcoat Composition described above including 0.01% by weight, based upon the total weight of the clearcoat composition, of Fluorad FC-430 used as a morphing additive.

2k Urethane Clearcoat Composition is product code E10CG066 commercially available from BASF Corporation.

Si Modified 2k Urethane Clearcoat Composition is the Acrylic Melamine Clearcoat Composition described above including 0.05% by weight, based upon the total weight of the clearcoat composition, of Byk-306 used as a morphing additive.

Low Gloss Carbamate #3 Clearcoat Composition is product code R10CG060Z commercially available from BASF Corporation, including a gloss reduction agent added thereto.

The Examples and Comparative Examples prepared as described above (and plasma treated as described above) were subjected to a series of tests to determine acid etch resistance, scratch resistance, and gloss retention. Acid etch resistance was measured in accordance with ASTM D-7356 (400 hr), with results presented as a standard “etch rating” and with lower values corresponding to higher etch resistance. Scratch resistance was measured in accordance with the Crockmeter method using 9 micron paper. Gloss was measured using a Micro Tri-Gloss model 4520 gloss meter commercially available from Byk-Gardner. The gloss meter is capable of producing gloss measurements at 20, 60, and 85 degrees. The results of the various tests are set forth below in Table 2, with gloss taken at 20°.

TABLE 2

| | Etch | Initial Gloss, | Final Gloss, | |
|---|---|---|---|---|
| Example | Rating | 20° | 20° | Gloss Retention, % |
| Comp. Ex. 1 | 10 | 87 | 69 | 79 |
| Ex. 1 | 9 | 54 | 52 | 96 |
| Ex. 2 | 8 | 87 | 80 | 92 |
| Ex. 3 | 10 | 87 | 80 | 92 |
| Ex. 4 | 10 | 64 | 87 | 73 |
| Comp. Ex. 2 | 7 | 86 | 67 | 77 |
| Ex. 5 | 6 | 54 | 52 | 96 |
| Ex. 6 | 7 | 86 | 63 | 73 |
| Ex. 7 | 6 | 88 | 82 | 93 |
| Ex. 8 | 7 | 87 | 67 | 77 |
| Comp. Ex. 3 | 7 | 88 | 63 | 71 |
| Ex. 9 | 8 | 52 | 50 | 96 |
| Ex. 10 | 5 | 87 | 71 | 81 |
| Ex. 11 | 7 | 85 | 52 | 61 |
| Comp. Ex. 4 | 4 | 84 | 42 | 50 |
| Ex. 12 | 4 | 61 | 51 | 83 |
| Ex. 13 | 2 | 81 | 53 | 65 |
| Ex. 14 | 3 | 84 | 59 | 70 |
| Comp. Ex. 5 | 6 | ** | ** | ** |
| Ex. 15 | 5 | ** | ** | ** |

** No scratch testing performed.

As made clear from the results obtained after testing the Examples and Comparative Examples, the Examples that were treated with a plasma treatment with plasma coating 1 and 2 consistently exhibited higher etch resistance, scratch resistance, and % gloss retention than untreated Comparative Examples (i.e., Comparative Examples 1-5). Likewise, the Examples that were treated with a plasma treatment in the absence of a precursor also consistently exhibited higher etch resistance, scratch resistance, and % gloss retention than untreated Comparative Examples. While results were mixed when silicon and fluorine-modified clearcoat compositions were treated with plasma coating 2, these Examples also showed consistently higher scratch resistance and gloss retention as compared to untreated Comparative Examples.

Obviously, many modifications and variations of the present invention are possible in light of the above teachings, and the invention may be practiced otherwise than as specifically described within the scope of the appended claims.

What is claimed is:

1. A technique of treating an automotive coating to modify surface-specific physical properties thereof while retaining bulk physical properties of the automotive coating, said method comprising the step of:
generating a plasma discharge in a plasma generating assembly; and
treating the automotive coating with the plasma discharge;
wherein the automotive coating is completely cured prior to treatment with the plasma discharge; and
wherein 1) a precursor material is introduced into the plasma discharge to form a thin film on the automotive coating, or 2) the plasma discharge is free of a precursor material and weak bonds are destroyed on or near the surface of the automotive coating.

2. A technique as set forth in claim 1 wherein the plasma discharge is generated under conditions of atmospheric pressure.

3. A technique as set forth in claim 1 wherein the plasma discharge is directed out of the assembly and onto the automotive coating to be treated.

4. A technique as set forth in claim 1 wherein the plasma generating assembly includes at least one pair of parallel electrodes spaced from each other and wherein an inert gas is introduced between the at least one pair of electrodes and an electrical current is passed between the at least one pair of electrodes to form the plasma discharge.

5. A technique as set forth in claim 4 wherein the precursor material is introduced between the at least one pair of electrodes along with the inert gas.

6. A technique as set forth in claim 5 wherein the precursor material is mixed with the inert gas prior to introducing the inert gas between the two electrodes.

7. A technique as set forth in claim 5 wherein free radical polymerization of molecules in the precursor material is initiated in the plasma discharge.

8. A technique as set forth in claim 5 wherein the precursor material is melted into a molten form in the plasma discharge.

9. A technique as set forth in claim 5 wherein the automotive coating is treated with the plasma discharge including the precursor material for a sufficient period of time to form the thin film on the automotive coating having a film thickness of from about 5 to about 600 nm.

10. A technique as set forth in claim 1 wherein the plasma discharge is free of the precursor material and weak bonds are destroyed on or near the surface of the automotive coating to modify the surface of the automotive coating itself.

11. A technique as set forth in claim 10 wherein the automotive coating is modified at a distance of from the surface of the automotive coating to a depth of less than five microns.

12. A technique as set forth in claim 10 wherein the automotive coating retains at least 10% higher gloss than similar untreated automotive coatings.

13. A technique as set forth in claim 10 wherein an etch ratings of the automotive coating is decreased by about one unit as compared to similar untreated automotive coatings.

14. A technique as set forth in claim 1 wherein the automotive coating comprises the reaction product of at least a resin component and a crosslinking agent that is reactive with the resin component.

15. A technique as set forth in claim 1 wherein the automotive coating comprises the reaction product of a dual cure coating composition comprising at least four components:
a radiation curable resin component (a1) that polymerizes upon exposure to actinic radiation,
a thermally curable binder component (a2) that polymerizes upon exposure to heat,
a thermally curable crosslinking component (a3) that has at least 2 isocyanate groups per molecule, and
at least one additive (a4) for absorbing or otherwise preventing transmission of ultraviolet radiation

16. A technique as set forth in claim 15 wherein the radiation-curable resin component (a1) is further defined as a dual-hydroxy carbamate-functional acrylate resin.

17. A technique as set forth in claim 15 wherein the radiation-curable resin component (a1) comprises a urethane(meth)acrylate.

18. A technique as set forth in claim 15 wherein the thermally curable binder component (a2) comprising at least two isocyanate-reactive groups.

19. A technique as set forth in claim 15 wherein the thermally curable crosslinking component (a3) comprises a blocked or unblocked di- and/or polyisocyanate.

20. A technique as set forth in claim 15 wherein the dual cure coating composition further comprises a photoinitiator.

21. A technique as set forth in claim 15 wherein the dual cure coating composition further comprises at least one thermal crosslinking initiator that forms radicals at a temperature of from 80° C. to 120° C.

22. A technique as set forth in claim 1 wherein the automotive coating comprises a morphing additive that is activated upon exposure to the plasma discharge and wherein the plasma discharge is free of a precursor material.

23. A technique as set forth in claim 22 wherein the automotive coating is modified at a distance of from the surface of the automotive coating to a depth of less than five microns.

24. A technique as set forth in claim 22 wherein the morphing additive is selected from the group of silicones, fluoropolymers, polyesters, acrylics, and combinations thereof.

25. A technique as set forth in claim 1 wherein the curable coating composition comprises a morphing additive present in an amount of from 0.005% to 0.5% based on the total weight of the curable coating composition.
